(19) United States
(12) Patent Application Publication
(10) Pub. No.: US 2026/0126375 A1
(43) Pub. Date: May 7, 2026

(54) RAPID DEAMIDATION SCREENING

(71) Applicant: PROTEIN DYNAMIC SOLUTIONS, INC., Wakefield, MA (US)

(72) Inventor: Belinda PASTRANA-RIOS, Wakefield, MA (US)

(73) Assignee: PROTEIN DYNAMIC SOLUTIONS, INC., Wakefield, MA (US)

(21) Appl. No.: 19/427,452

(22) Filed: Dec. 19, 2025

Related U.S. Application Data
(63) Continuation of application No. PCT/US2024/034653, filed on Jun. 20, 2024.
(60) Provisional application No. 63/509,340, filed on Jun. 21, 2023.

(51) Int. Cl. G01N 21/27; G01N 21/17; G01N 33/68

(52) U.S. Cl. CPC G01N 21/27; G01N 33/6854; G01N 2021/1748; G01N 2021/1765; G01N 2201/121; G01N 2440/16

(57) ABSTRACT

Rapid comparability assessment of asparagine or glutamine deamidation of therapeutic proteins, peptides other modalities against a reference standard using two trace two-dimensional correlation spectroscopy. Asynchronous plots are generated providing information of the abundance of the components under evaluation. The method is valid for an array of protein samples providing an estimate of the extent of deamidation. The results of which are orthogonal to HPLC cation exchange, thus validating the existence of the deamidated species and if aggregate is present. This rapid method run time is only 10 minutes for up to 21 samples in their formulation condition.

### RELATED APPLICATION

This application is a continuation of International Application No. PCT/US2024/034653, filed on Jun. 20, 2024, which claims the benefit of U.S. Provisional Patent Application No. 63/509,340, filed on Jun. 21, 2023, the entire contents of which are incorporated herein by reference.

### STATEMENT REGARDING FEDERALLY SPONSORED RESEARCH

This invention was made with Government support under Award No. 1632420 awarded by the National Science Foundation. The Government has certain rights in this invention.

### FIELD OF THE INVENTION

The present invention relates to a system and method for rapid screening of deamidated proteins in a sample by comparing to a reference standard. The system and method can screen for deamidated proteins independent of the modality. Moreover, the system and method can be used to screen both drug substances (DS) and drug products (DP) for deamidated proteins, and the testing for protein deamidation using the system and method can be assessed for samples varying in formulation and samples that are under or were subjected to forced degraded conditions providing a highly flexible method for developability, comparability and forensic evaluations.

### BACKGROUND

The number of biologics that have been approved by regulatory authority has exceeded small molecule therapeutics for the treatment of diseases.^{1,2} The Biopharma industry will always require analytical tools that provide rapid, robust and reproducible results to accelerate speed to market, while ensuring safety and efficacy of the therapeutic protein to benefit the patient. Deamidation is a critical quality attribute for both drug substances (DS), as well as drug products (DP). Deamidation is a non-enzymatic degradation pathway for proteins comprised of aqueous solvent exposed asparagine and glutamine residues.^{3,4} This process occurs in proteins at high pH, low pH, under thermal stress or combination of these conditions. This is a critical event that introduces a negative charge in the sequence of the protein and if it occurs within the binding interface of the target can lead to loss of specificity, affinity and even efficacy. Also, if the negative charge is localized within the Fc region of an antibody, then it may limit the effector function involving the antibody dependent cellular cytotoxicity (ADCC). Furthermore importantly, asparagine and glutamine deamidation may lead to loss of stability, aggregation and immunogenicity.

However, current methods of evaluating an array of therapeutic proteins with respect to deamidation quality attributes are time consuming, and may further depend on factors such as size of the sample and modality. Moreover, these methods typically require time-consuming sample preparation before any evaluation can be performed. What is needed is a system and method for reliably evaluating an array of therapeutic proteins in a short time period to provide an estimate of deamidation.

### SUMMARY OF THE INVENTION

The system and method described herein provide a rapid method of screening an array of therapeutic proteins against one or more reference standards under controlled temperature conditions in a short time period, such as 10 minutes, to determine an estimate of the presence of deamidated proteins. This short timeframe is for the evaluation of a plurality of samples against a predetermined number of references, independent of size and modality. For example, the system and method may evaluate 21 samples against 2 references independent of size or modality in a 10 minute time period. Vibrational spectroscopy, such as infrared spectroscopy which is highly selective and sensitive, may be used to evaluate the samples. In particular, after spectral images are obtained suing vibrational spectroscopy, two-trace two-dimensional correlation spectroscopy can be applied to provide a comparative analysis between a sample protein against one or more reference standards at a controlled temperature. The controlled temperature may be in the range of range of 20-65° C., although the deamidation screening method will be routinely implemented at 25° C. Based on the comparisons, the system and method provides an estimate of asparagine and glutamine deamidation for therapeutic proteins and peptides. The system and method may also be used for screening related to Adeno-associated virus and cell and gene therapy. In addition, the system and method can be used to screen for exosomes, which may be carriers for therapeutics.

To evaluate samples, the system and method use a vibrational spectroscopy apparatus, such as a quantum cascade laser (QCL) transmission microscope, Raman spectroscopy and microscopy, FT-IR, or light detection and ranging (LIDAR) system, with a slide cell array and dedicated software that provide rapid deamidation screening. The slide cell array may be comprised of a polymer, such as polyethylene, salt such as calcium fluoride or barium fluoride, or other polymer material that is transparent within most of the spectral region of interest. The samples to be screened, along with potentially one or more controls, are provided within the cells of the slide cell array, and the QCL is used to acquire images of the slide cell array. The system may be implemented as a platform, with data from the QCL provided to the dedicated software for analysis of the samples. The QCLs provide real-time hyperspectral (HS) image acquisition with enhanced signal to noise ratio. The QCL contains a light source, and the intensity of the light source may be comparable to the synchrotron and is non-destructive. Each hyperspectral image obtained is comprised of 223,000 spectra. The hyperspectral images may be acquired within the spectral region of 1775-1435 cm⁻¹ at a spectral resolution of 4 cm⁻¹. As mentioned, the hyperspectral images may also be acquired by other vibrational spectroscopy apparatus, such as a Raman amplifier or LIDAR.

Dedicated software allows for the processing of the series of the acquired spectra for a linear baseline correction, and a two-trace two-dimensional (2T2D) correlation algorithm is applied for the deamidation assessment of all samples within the array. Two-trace two-dimensional synchronous and asynchronous plots are generated with provide the spectral differences between reference and the sample, and key signature peaks associated with a deamidation process are evaluated to determine an estimate of deamidation. For example, the plots can be assessed to determine if a peak is present at a wavenumber associated with associated with glutamine or asparagine deamidation, or with glutamate or aspartate deamidation. In particular, if a positive peak is present at certain wavenumbers, then it is determined that there was deamidation. Peak ratios are also used to determine estimates of the presence of deamidated proteins in the samples.

### Deamidation Screening

For example, a method for performing deamidation screening of a sample may comprise:
a) providing the sample in a slide containing at least one sample well and at least one reference well;
b) acquiring at least one spectral image of the sample and at least one spectral image of the reference using a quantum cascade laser microscope under a controlled temperature condition;
c) identifying and selecting, in at least one of the acquired spectral images of the sample and the reference, a region of interest;
d) obtaining spectral data for the sample for the sample region of interest and obtaining spectral data of the reference for the reference region of interest;
e) applying a baseline correction to the spectral data for the sample and the reference for the region of interest;
f) applying a two-trace two-dimensional correlation to the baseline corrected sample and reference spectral data to generate a synchronous spectrum Φ(v₁, v₂) and asynchronous spectrum Ψ(v₁, v₂);
generating a synchronous plot and an asynchronous plot from the synchronous and asynchronous spectra; and
analyzing signature peaks to determine an estimation of deamidation of the sample.

The region of interest may be the entire field of view, or a subset of the field of view.

The method may further comprise generating weighted difference spectra for the sample and reference, and comparing the weighted difference spectra to determine an estimate of deamidation. And, the weighted difference spectra may be evaluated to determine if the deamidated sample is also aggregated.

In the method, acquiring at least one spectral image of the sample using a quantum cascade laser microscope includes acquiring at least one first hyperspectral image of the sample.

The method may further comprise applying a 2T2D correlation coefficient ρ(v₁, v₂) to the synchronous spectrum, and applying a disrelation coefficient ξ(v₁, v₂) to the asynchronous spectrum, as given by:

$$ρ ( {v}_{1} , {v}_{2} ) = Φ ( {v}_{1} , {v}_{2} ) / \sqrt{Φ ( {v}_{1} , {v}_{1} ) \cdot Φ ( {v}_{2} , {v}_{2} )}$$

$$ξ ( {v}_{1} , {v}_{2} ) = Ψ ( {v}_{1} , {v}_{2} ) / \sqrt{Φ ( {v}_{1} , {v}_{1} ) \cdot Φ ( {v}_{2} , {v}_{2} )}$$

where:

$${ρ ( {v}_{1} , {v}_{2} )}^{2} + {ξ ( {v}_{1} , {v}_{2} )}^{2} = 1 .$$

### BRIEF DESCRIPTION OF THE DRAWINGS

FIG. 1A is an example of a 2T2D asynchronous plot for the therapeutic protein pembrolizumab IgG4 as compared to a reference sample.

FIG. 1B is an example of a 2T2D asynchronous plot for the therapeutic protein briakinumab IgG4 as compared to a reference sample.

FIG. 1C is an example of a 2T2D asynchronous plot for the therapeutic protein evolocumab IgG4 as compared to a reference sample.

FIG. 1D is an example of a 2T2D asynchronous plot for the therapeutic protein trastuzumab IgG4 as compared to a reference sample.

FIG. 2A is an example of a cation exchange (CEx) chromatogram for a reference sample of pembrolizumab IgG4.

FIG. 2B is an example of a cation exchange (CEx) chromatogram for a sample of pembrolizumab IgG4 under forced degradation conditions.

FIG. 3A is an illustration of estimated difference spectra generated based on 2T2D correlation analysis within the spectral region 1775-1435 cm−1 for pembrolizumab IgG4 reference/control and the forced degraded samples.

FIG. 3B is an illustration of an estimated sample spectrum for the forced degraded samples of FIG. 3A.

FIG. 3C is an illustration of an estimated sample spectrum for the control/reference sample of FIG. 3A.

FIG. 4A is an example of a cation exchange (CEx) chromatogram for a reference sample of briakinumab IgG4.

FIG. 4B is an example of a cation exchange (CEx) chromatogram for a sample of briakinumab IgG4 under forced degradation conditions.

FIG. 5A is an illustration of a spectral overlay within the spectral region 1775-1435 cm⁻¹ for a reference and the forced degraded sample of briakinumab IgG4.

FIG. 5B is an illustration of a 2T2D weighted reference spectra for the reference sample of briakinumab IgG4.

FIG. 5C is an illustration of a 2T2D weighted sample spectra for the forced degraded sample of briakinumab IgG4.

FIG. 6A is an example of a cation exchange (CEx) chromatogram for a reference sample of evolocumab IgG4.

FIG. 6B is an example of a cation exchange (CEx) chromatogram for a sample of evolocumab IgG4 under forced degradation conditions.

FIG. 7A is an illustration of a spectral overlay within the spectral region 1775-1435 cm⁻¹ for a reference and the forced degraded sample of evolocumab IgG4.

FIG. 7B is an illustration of a 2T2D weighted reference spectra for the reference sample of evolocumab IgG4.

FIG. 7C is an illustration of a 2T2D weighted sample spectra for the forced degraded sample of evolocumab IgG4.

FIG. 8A is an example of a cation exchange (CEx) chromatogram for a reference sample of trastuzumab IgG4.

FIG. 8B is an example of a cation exchange (CEx) chromatogram for a for a sample of trastuzumab IgG4 under forced degradation conditions.

FIG. 9A is an illustration of a spectral overlay within the spectral region 1775-1435 cm⁻¹ for a reference and the forced degraded sample of trastuzumab IgG4.

FIG. 9B is an illustration of a 2T2D weighted reference spectra for the reference sample of trastuzumab IgG4.

FIG. 9C is an illustration of a 2T2D weighted sample spectra for the forced degraded sample of trastuzumab IgG4.

FIG. 10 is an illustration of flow diagrams for deamidation screening of a plurality of samples.

### DETAILED DESCRIPTION

The system and method described herein provide for a rapid process for screening of deamidated proteins in a sample by comparison against a reference standard of the sample.

### Deamidation Screening Set-Up

The system and method subject a series of therapeutic protein samples in their formulations to a comparative analysis with one or more reference standards at a controlled temperature. The controlled temperature may be in the range of, for example, 20-65° C., although the deamidation screening method may be routinely implemented at 25° C. A slide cell array may be provided that includes a plurality of wells. The slide cell array may be comprised of a polymer, such as polyethylene, calcium fluoride, barium fluoride, or other polymer that is transparent within most of the spectral region of interest. Samples to be screened for deamidation and appropriate reference, or control, samples are placed in predefined wells within the slide array. Negative controls containing no protein or formulation may also be included in one or more wells in the slide array. Only a small amount of a sample, such as 1 μL, per well is required for analysis. After the samples, reference samples, and any negative controls are placed in the slide, the slide cell is then covered and assembled into a thermally controlled slide cell holder to ensure a controlled thermal environment. The slide cell holder accessory may be, for example, a controllable heated chamber configured to receive the slide cell.

### Hyperspectral Image Acquisition

After the slide cell array is placed in the slide cell holder, spectral images of the slide cell array are obtained. These spectral images may be obtained using real-time hyperspectral (HS) image acquisition by a Quantum Cascade Laser Microscope (QCLM). The QCLM HS image acquisition may be carried out for all samples, negative controls and reference standard within the slide cell array at a one or more defined temperatures. For example, the HS image acquisition may be conducted at a defined temperature of 25±0.1° C. The HS images may be, for example, comprised of 223,000 QCLM spectra within the spectral region of 1775-1435 cm⁻¹ at a spectral resolution of 4 cm⁻¹. Dedicated software allows for the processing of the series of QCL IRM spectra for a linear baseline correction, and a two-trace two-dimensional correlation algorithm is applied for the deamidation assessment of all samples within the array. Other microscopy techniques may be applied to obtain the spectral images, such as FT-IR or Raman microscopy. However, the enhanced signal to noise ratio, fast HS image acquisition and non-destructive nature of the light source obtained using the QCLM enable the small sample requirements that benefit the biopharma industry in sample management. Before the spectral data is further processed, the system may perform a baseline correction to the spectral data for one or more regions of interest. In particular, there is typically scatter, which causes a deviation of the base of the spectrum, which may appear as curvature of the baseline. The baseline correction removes this scatter. The baseline correction may be a spline correction that removes the curvature.

### 2T2D Correlation Analysis

After the spectral images are acquired and spectral data obtained from the spectral images baseline corrected, a two-trace two-dimensional (2T2D) correlation^{5,6} process is applied to the spectral data. From a pair of spectra a two trace two-dimensional correlation can be applied to generate a synchronous spectrum Φ(v₁, v₂) and asynchronous spectrum Ψ(v₁, v₂). The synchronous spectrum Φ(v₁, v₂) and asynchronous spectrum Ψ(v₁, v₂) are given by:

$$Φ ( {v}_{1} , {v}_{2} ) = \frac{1}{2} [ s ( {v}_{1} ) \cdot s ( {v}_{2} ) + r ( {v}_{1} ) \cdot r ( {v}_{2} ) ] \qquad ( 1 )$$

$$Ψ ( {v}_{1} , {v}_{2} ) = \frac{1}{2} [ s ( {v}_{1} ) \cdot r ( {v}_{2} ) - r ( {v}_{1} ) \cdot s ( {v}_{2} ) ] \qquad ( 2 )$$

The first, original spectra, s(v), corresponds to the sample and the second spectra, r(v), corresponds to the reference, respectively. A 2T2D correlation coefficient is then applied ρ(v₁, v₂) to the synchronous evaluation, and a disrelation coefficient ξ(v₁, v₂) to the asynchronous evaluation resulting in the scaled version of the 2T2D correlation spectra, as given by:

$$ρ ( {v}_{1} , {v}_{2} ) = Φ ( {v}_{1} , {v}_{2} ) / \sqrt{Φ ( {v}_{1} , {v}_{1} ) \cdot Φ ( {v}_{2} , {v}_{2} )} \qquad ( 3 )$$

$$ξ ( {v}_{1} , {v}_{2} ) = Ψ ( {v}_{1} , {v}_{2} ) / \sqrt{Φ ( {v}_{1} , {v}_{1} ) \cdot Φ ( {v}_{2} , {v}_{2} )} \qquad ( 4 )$$

Where:

$${ρ ( {v}_{1} , {v}_{2} )}^{2} + {ξ ( {v}_{1} , {v}_{2} )}^{2} = 1 \qquad ( 5 )$$

This indicates the complementarity nature of the quantities.

Two contour plots are generated. These are the synchronous (Φ(v₁, v₂)) plot where dominant spectral components of the samples: s(v) and r(v) are observed. The diagonal is comprised of auto peaks where v₁=v₂. The cross peaks are always positive. The second plot is the asynchronous (Ψ(v₁, v₂)) plot, which is more informative. In the case Ψ>0, then the intensity contribution of the functional group is from v₁, corresponding to the first component being more abundant. In the case Ψ<0, then the intensity contribution of the functional group is from v₂, therefore, the second component of the sample is more abundant. Also, this indicates that peaks of the same intensity and sign correspond to the same component within the sample.

### Deamidation Screening Assessment

The synchronous and asynchronous 2T2D plots are then used to conduct a deamidation screening assessment of the samples. An estimate of the extent of deamidation is obtained when compared to the reference standard at a predefined temperature, such as room temperature (around 25° C.). The determination is based on the intensity of key signature peaks known to be associated directly with deamidation, such as glutamine and asparagine deamidation. The result will be defined as pass or fail based on a threshold value, such as a value defined by the Chemistry Manufacturing and Controls (CMC) for the product quality attribute and confidence limits set at 95%.

### Orthogonality to HPLC Cation Exchange (CEx) Chromatographic Methods

To determine the accuracy and reliability of the rapid deamidation screening using the systems and methods implementing the 2T2D correlation process and analysis described herein, an evaluation of the orthogonality of the deamidation screening described herein with known high speed liquid chromatography (HPLC) CEx methods for the same samples was performed. Both qualitative and quantitative evaluations were performed, providing proof of orthogonality for 4 different IgG4 scaffold engineered clinical antibodies under control and forced degraded conditions. For the comparative analysis, the control sample was defined as the reference. The therapeutic protein samples under control conditions (reference) were in phosphate buffered saline at pH 7.4 and the forced degraded sample was in 20 mM Tris, 10 mM EDTA at pH 8.5. For the QCLM deamidation screen method using the 2T2D correlation analysis described herein, the samples and reference were analyzed as-is in their formulation conditions. That is, no pre-processing of the samples was performed. Meanwhile, for the HPLC CEx method, the samples and reference were diluted five-fold using a mobile phase buffer. The HPLC CEx method required a different mobile phase than that of the therapeutic protein formulation. The mobile phase was comprised of: (A) 20 mM MES at pH 6.2 and (B) 20 mM MES, 150 mM NaCl at pH 6.2 to generate the gradient for elution of the desired therapeutic protein. Finally, chromatographic methods require single sample injection at a time and the elution times are dependent on the protein's physical and chemical properties. As seen in Table 1 below, comparatively, the QCLM results were within 3-7% in agreement with the HPLC CEx results. The QCLM method does not involve any change in formulation and therefore remains true to the therapeutic protein sample under investigation minimizing variability in the results obtained. This is not the case with HPLC CEx method when the buffer conditions must be changed to that of the mobile phase, potentially leading to aggregation of the protein and resulting in reduced detection of the protein and its deamidated species. The results of the comparison between the deamidation screening process, such as using QCLM, described herein as compared to results from HPLC CEx methods are provided in Table 1 below:

TABLE 1
Summary of QCLM and HPLC cation
exchange chromatography results

% Extent of Deamidation

| mAb | QCLM^{a} | HPLC CEx^{b} |
|---|---|---|
| trastuzumab IgG4 | 92.4 | 89.5 |
| evolocumab IgG4 | 76.2 | 72.4 |
| pembrolizumab IgG4 | 62.8 | 56.2 |
| briakinumab IgG4 | 11.4 | 15.3* |

^{a}Reference and samples were evaluated in their original buffer conditions.
^{b}Reference and samples were 5 fold diluted in 20 mM MES at pH 6.2 prior to injection.
*Higher salt gradient was used to try to elute the acidic peaks.

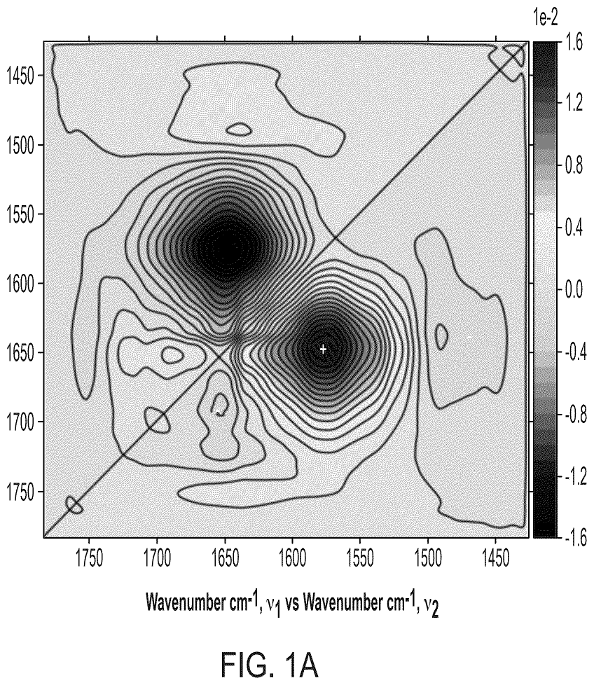

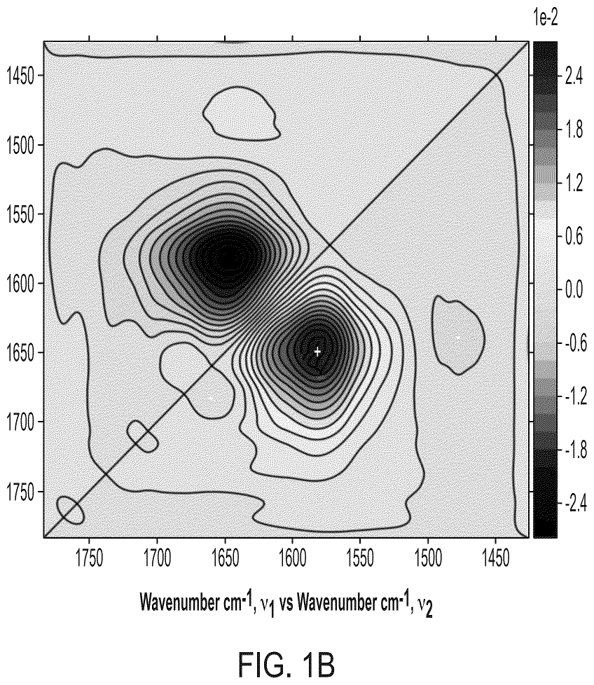

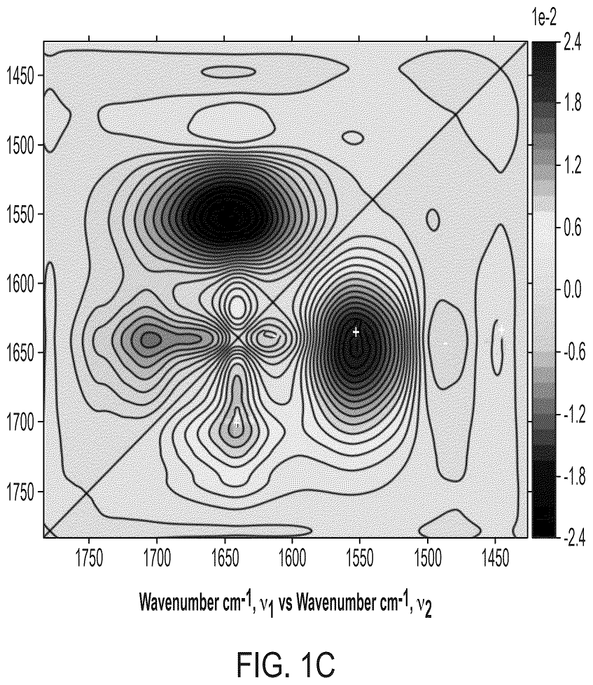

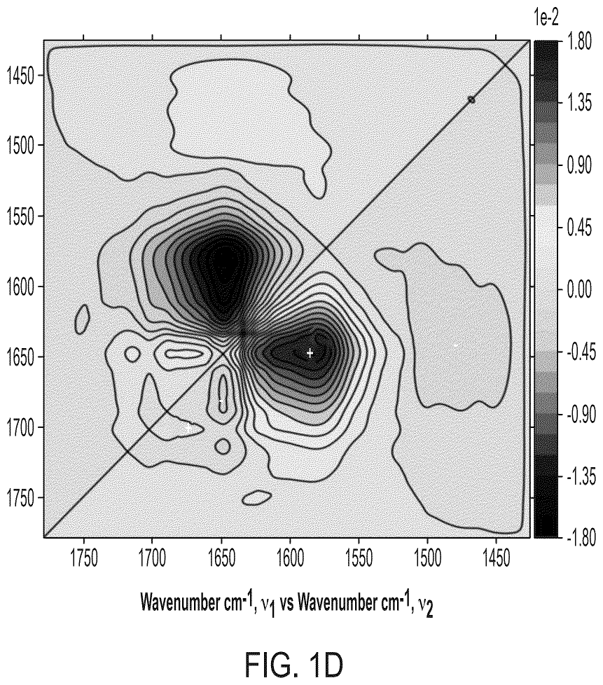

FIGS. 1A-D provide examples of 2T2D asynchronous plots for four therapeutic proteins, with FIG. 1A being the plot for pembrolizumab IgG4, FIG. 1B being the plot for briakinumab IgG4, FIG. 1C being the plot for evolocumab IgG4, and FIG. 1D being the plot for trastuzumab IgG4. The plot is symmetrical and the sign of the cross peaks has been identified on the bottom triangle sector. This is the face value of the intensity change for the cross peak of interest. It is a direct evaluation of the deamidation occurrence in the sample when compared to the reference. Also, the 2T2D asynchronous plot can provide evidence that the deamidation event led to loss of stability or aggregation of the sample.

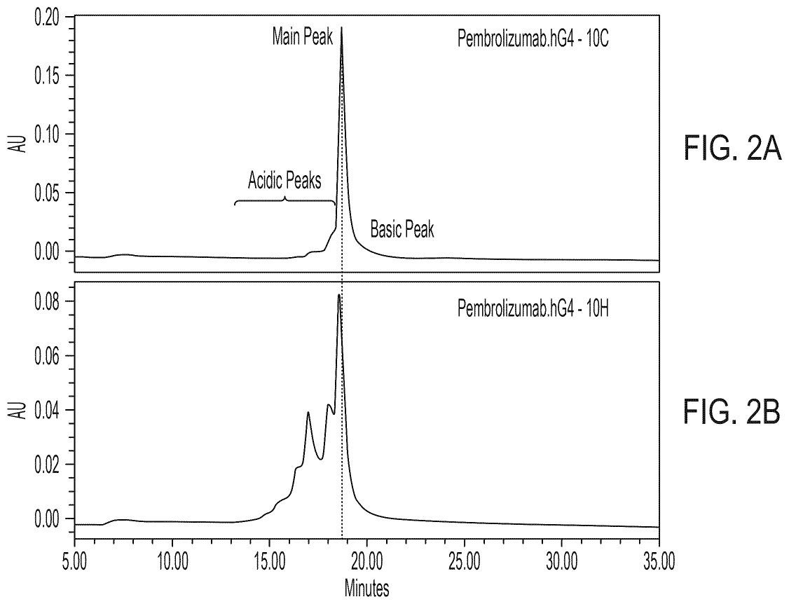

FIG. 2A provides an example of a CEx chromatogram for a reference sample of pembrolizumab IgG4, and FIG. 2B provides an example of a CEx chromatogram of a pembrolizumab IgG4 forced degradation sample. Peaks to the left of the dotted line show acid species, peaks to the right of the dotted line show basic species, the center peak is from the main species. The elution time for the main peak was around 18.8 minutes. That is, these peaks to either side of the main peak seen in FIG. 2B illustrate different forms of deamidation.

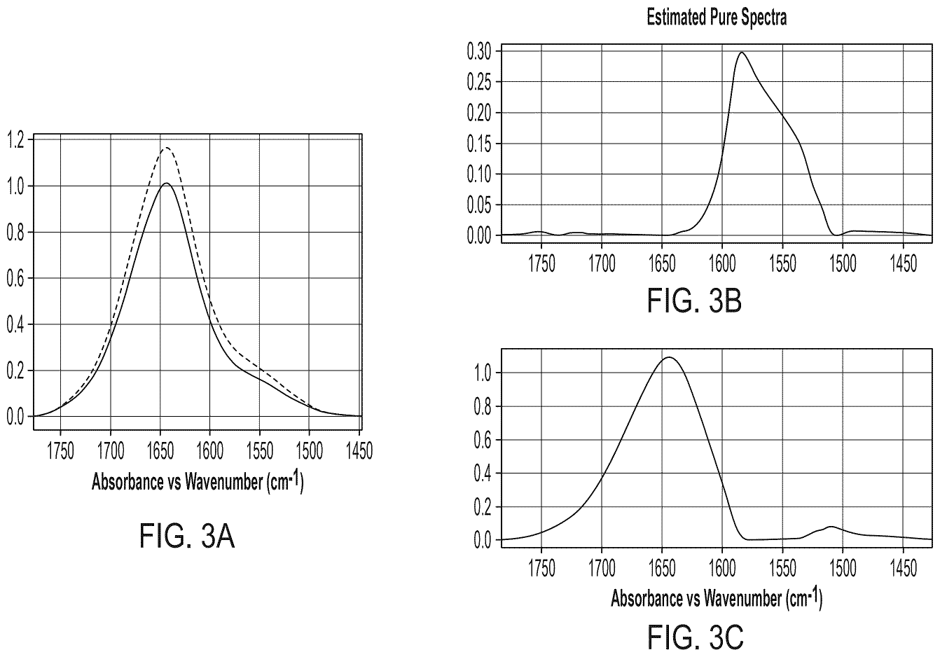

FIGS. 3A-C illustrate spectral overlays and estimated weighted spectra of pembrolizumab IgG4 for reference and forced degraded samples. FIG. 3A is an illustration of estimated difference spectra generated based on 2T2D correlation analysis within the spectral region 1775-1435 cm−1 for pembrolizumab IgG4 reference/control and the forced degraded samples. In FIG. 3A, the spectral overlay (top gray line) is the experimental sample spectrum, and the bottom black line corresponds to the experimental reference spectrum. FIG. 3B is an illustration of an estimated sample spectrum for the forced degraded samples of FIG. 3A. FIG. 3C is an illustration of an estimated sample spectrum for the control/reference sample of FIG. 3A. The estimated spectra of FIGS. 3A and 3B may be 2T2D weighted spectra. As can be seen, the shoulder in the line present between 1600 and 1500 cm⁻¹ in FIG. 3B represents deamidation.

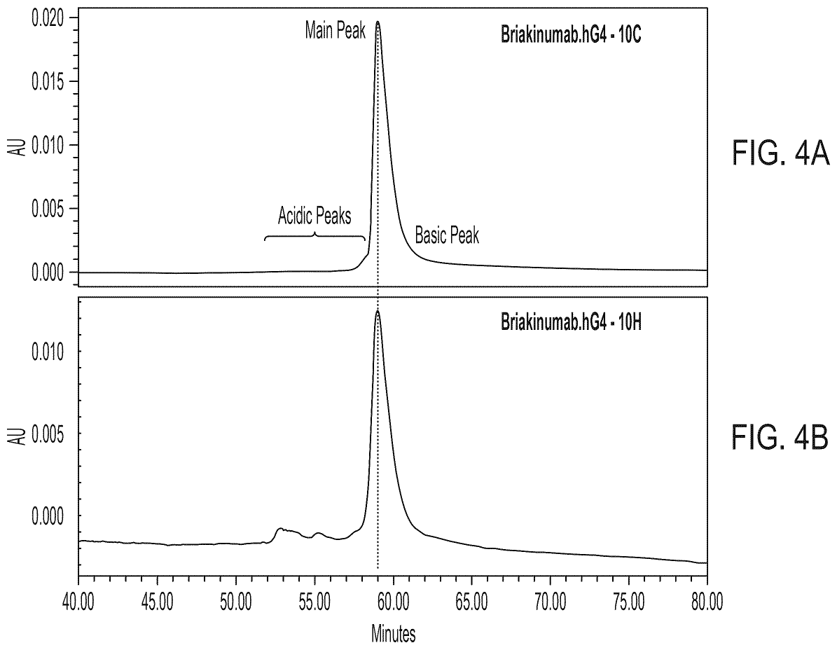

FIG. 4A provides an example of a CEx chromatogram for a reference sample of briakinumab IgG4, and FIG. 4B provides an example of a CEx chromatogram of a briakinumab IgG4 forced degraded sample. Peaks to the left of the dotted line show acid species, peaks to the right of the dotted line show basic species, the center peak is from the main species. The elution time for main peak was around 58 minutes. More importantly, although higher NaCl gradient was employed the deamidated species did not elute from the matrix effectively. At times, the chromatographic method causes unwanted aggregation of the deamidated species due to the buffer exchange during the run.

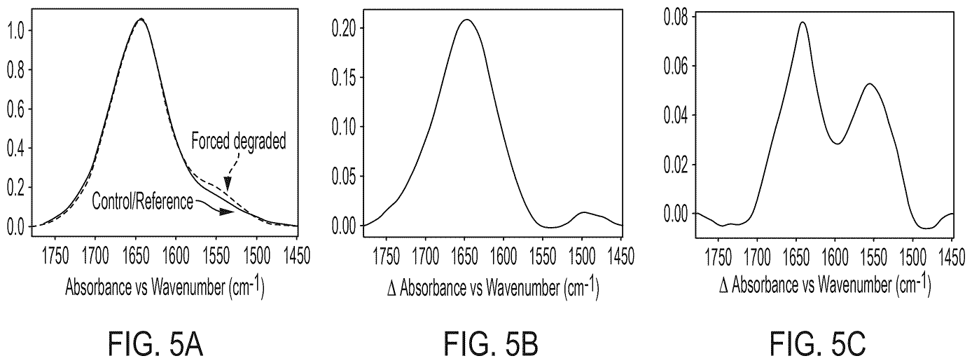

FIGS. 5A-C illustrate spectral overlays and estimated weighted spectra within the spectral region 1775-1435 cm⁻¹ for reference and forced degraded samples of briakinumab IgG4. FIG. 5A is an illustration of a spectral overlay within the spectral region 1775-1435 cm⁻¹ for a reference and the forced degraded sample of briakinumab IgG4. FIG. 5B is an illustration of a 2T2D weighted reference spectra for the reference sample of briakinumab IgG4. FIG. 5C is an illustration of a 2T2D weighted sample spectra for the forced degraded sample of briakinumab IgG4. As can be seen comparing FIGS. 5B and 5C, significant deamidation has occurred in the forced degraded sample when compared to the control/reference sample.

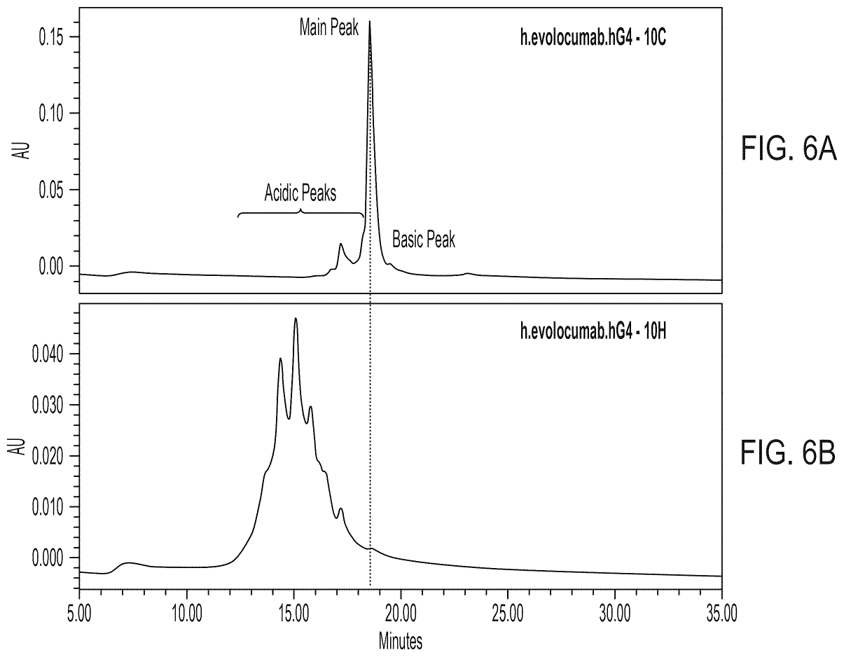

FIG. 6A provides an example of a CEx chromatogram for a reference sample of evolocumab IgG4, and FIG. 6B provides an example of a CEx chromatogram of a evolocumab IgG4 forced degraded sample. Peaks to the left of the dotted line show acid species, peaks to the right of the dotted line show basic species, the center peak is from the main species. The elution time for the main peak was observed at around 18.8 minutes for the control/reference sample.

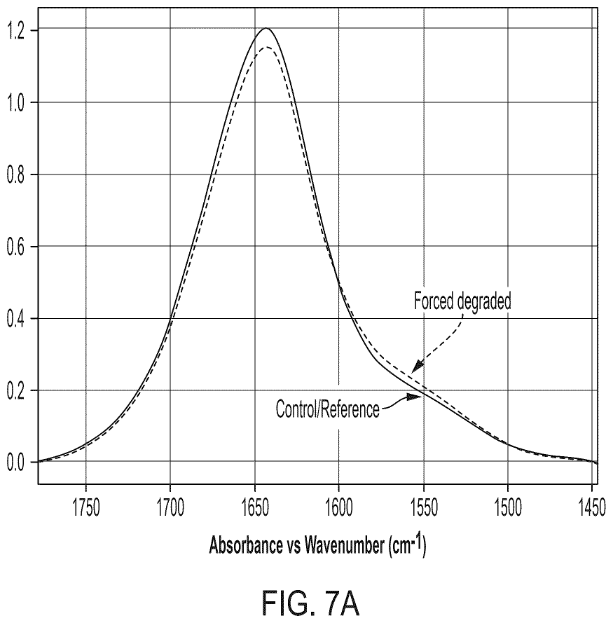

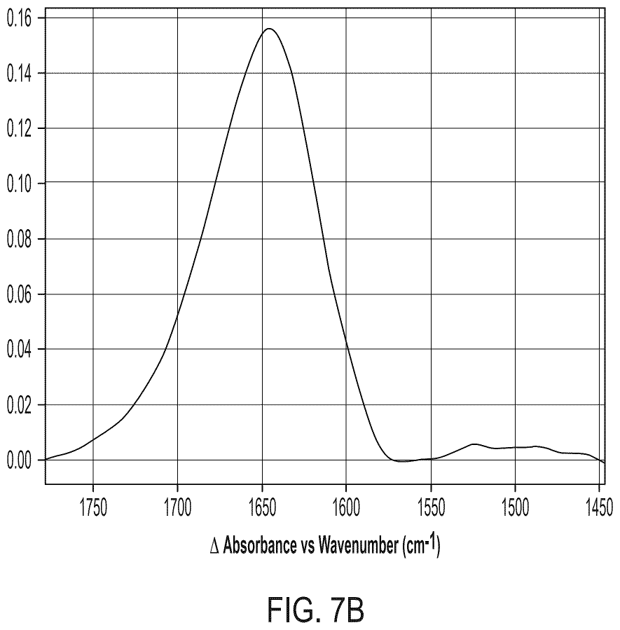

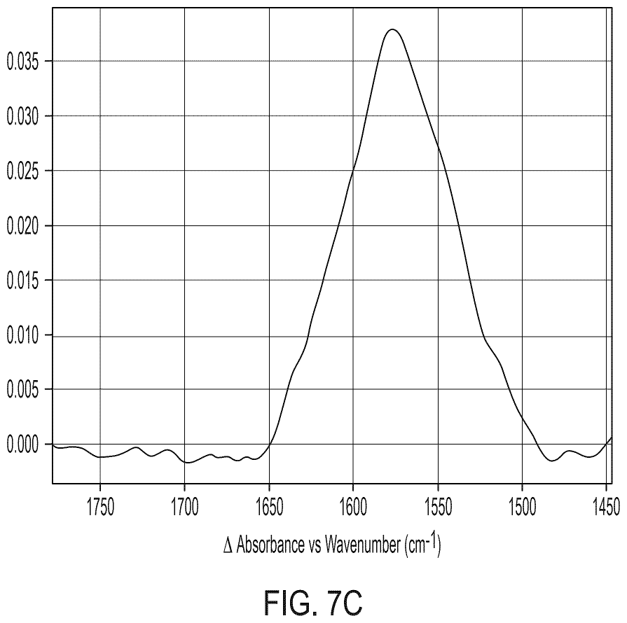

FIGS. 7A-C illustrate spectral overlays and estimated weighted spectra within the spectral region 1775-1435 cm⁻¹ for reference and forced degraded samples of evolocumab IgG4. FIG. 7A is an illustration of a spectral overlay within the spectral region 1775-1435 cm⁻¹ for a reference and the forced degraded sample of evolocumab IgG4. FIG. 7B is an illustration of a 2T2D weighted reference spectra for the reference sample of evolocumab IgG4. FIG. 7C is an illustration of a 2T2D weighted sample spectra for the forced degraded sample of evolocumab IgG4. As seen from the figures, the 2T2D deamidation screening method is sensitive to a deamidation event, aggregation presence, and can be used to verify the HPLC CEx results for the same sample.

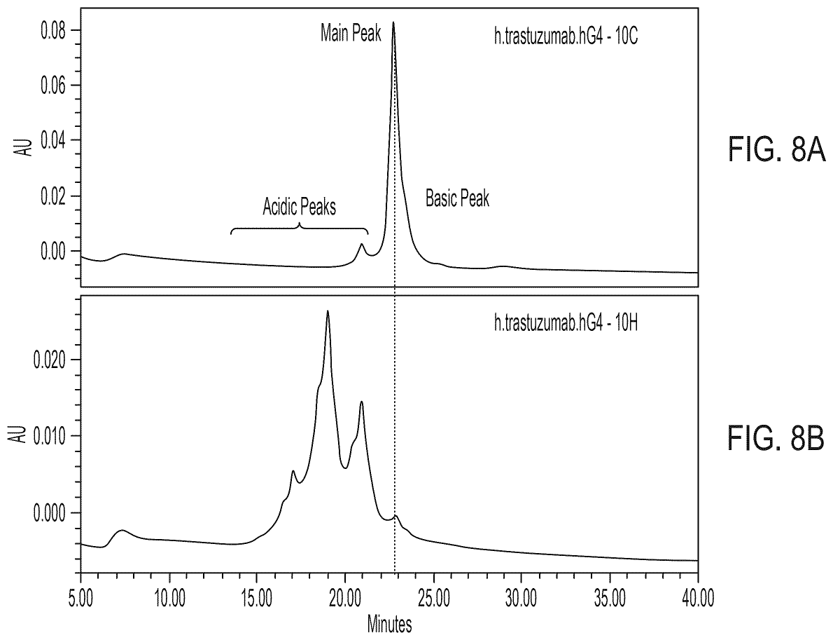

FIG. 8A provides an example of a CEx chromatogram for a reference sample of trastuzumab IgG4, and FIG. 8B provides an example of a CEx chromatogram of a trastuzumab IgG4 forced degraded sample. Peaks to the left of the dotted line show acid species, peaks to the right of the dotted line show basic species, the center peak is from the main species. The elution time of the main peak was around 23 min and the chromatogram for the forced degraded sample, as can be seen in FIG. 8B, is mainly comprised of acidic species.

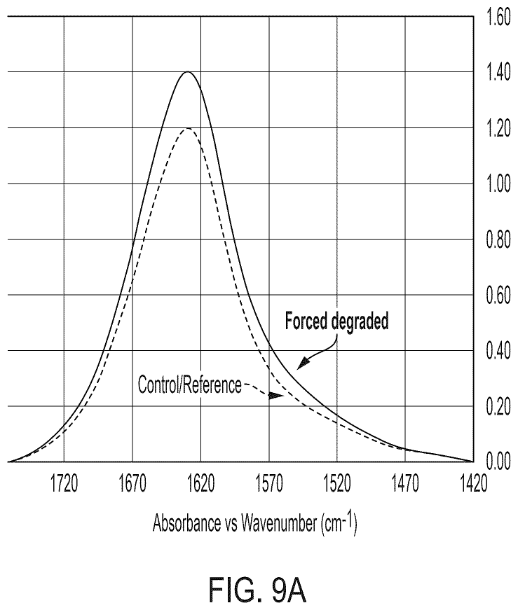

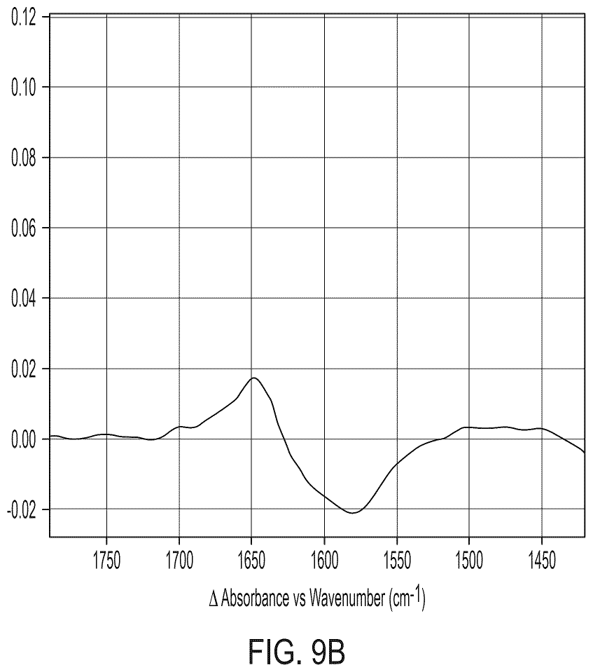

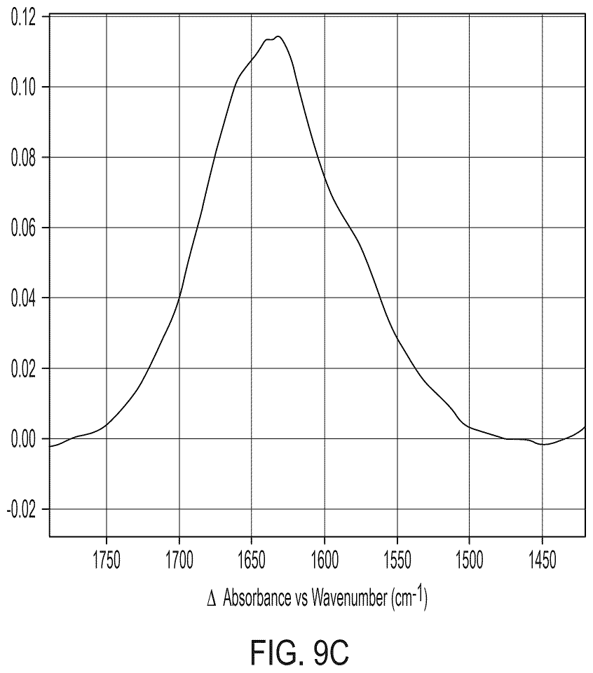

FIGS. 9A-C illustrate spectral overlays and estimated weighted spectra within the spectral region 1775-1435 cm⁻¹ for reference and forced degraded samples of Trastuzumab IgG4. under control and forced degraded conditions. FIG. 9A is an illustration of a spectral overlay within the spectral region 1775-1435 cm⁻¹ for a reference and the forced degraded sample of trastuzumab IgG4. FIG. 9B is an illustration of a 2T2D weighted reference spectra for the reference sample of trastuzumab IgG4. FIG. 9C is an illustration of a 2T2D weighted sample spectra for the forced degraded sample of trastuzumab IgG4.

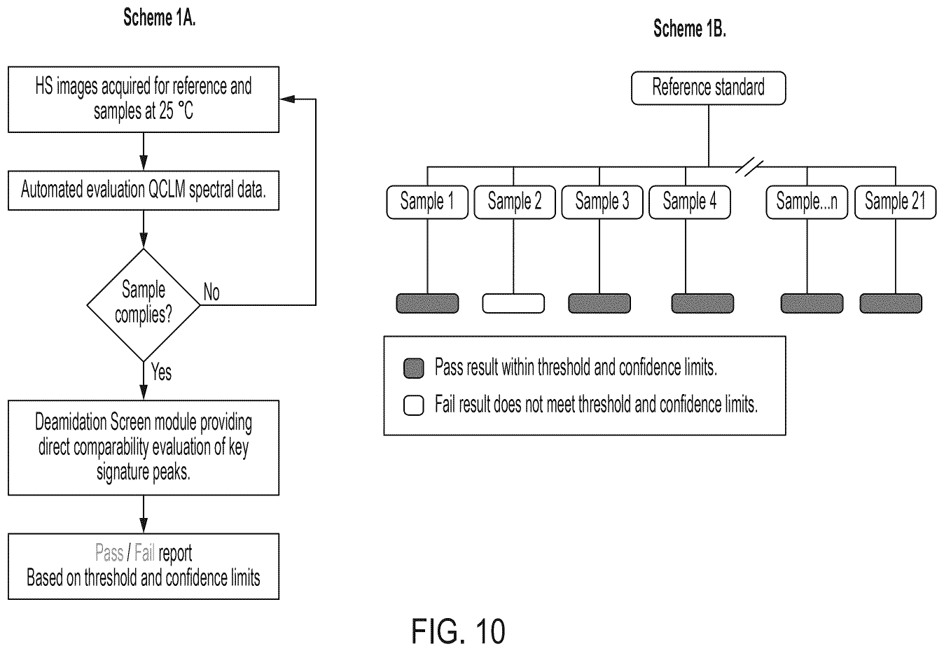

FIG. 10 illustrates the workflow of the therapeutic protein deamidation screening described herein, and comparability results to determine pass/fail results of screened samples. As shown in FIG. 10, Scheme 1A illustrates the process flow for the deamidation screening. As shown, HS images are acquired for a reference sample of sample to be screened of a selected protein. QCLM spectral data are acquired, and an automated evaluation is performed. A determination may be made to see if the sample complies with certain predefined quality control conditions, such as whether air bubbles are present. If the sample does not comply, then the process starts over and QCLM spectral data for new sample amounts are acquired. If the sample complies, then a deamidation screening module using 2T2D correlation processing as described above provides a direct comparability evaluation of key peaks. Based on the comparison, a pass/fail report is generated based on whether threshold and confidence limits on any determined deamidation are met. Scheme 1B represents an example of comparison results for deamidation screening performed on 21 samples as compared to a reference sample of a particular protein. This comparability assessment occurs in 10 minutes independent of modality and formulation conditions. The results are based on both critical quality attributes and product quality attributes.

1. A method for deamidation screening of a sample comprising:
a) providing the sample in a slide containing at least one sample well and at least one reference well;
b) acquiring at least one spectral image of the sample and at least one spectral image of the reference using a quantum cascade laser microscope under a controlled temperature condition;
c) identifying and selecting, in at least one of the acquired spectral images of the sample and the reference, a region of interest;
d) obtaining spectral data for the sample for the sample region of interest and obtaining spectral data of the reference for the reference region of interest;
e) applying a baseline correction to the spectral data for the sample and the reference for the region of interest;
f) applying a two-trace two-dimensional correlation to the baseline corrected sample and reference spectral data to generate a synchronous spectrum Φ(v₁, v₂) and Asynchronous Spectrum Ψ(v₁, v₂);
generating a synchronous plot and an asynchronous plot from the synchronous and asynchronous spectra; and
analyzing signature peaks to determine an estimation of deamidation of the sample.

2. The method of claim 1, further comprising generating weighted difference spectra for the sample and reference, and comparing the weighted difference spectra to determine an estimate of deamidation.

3. The method of claim 2, further comprising evaluating the difference spectra to determine if the deamidated sample is also aggregated.

4. The method of claim 1, wherein acquiring at least one spectral image of the sample using a quantum cascade laser microscope includes acquiring at least one first hyperspectral image of the sample.

5. The method of claim 1, further comprising applying a 2T2D correlation coefficient ρ(v₁, v₂) to the synchronous spectrum, and applying a disrelation coefficient ξ(v₁, v₂) to the asynchronous spectrum, as given by:
$$ρ ( {v}_{1} , {v}_{2} ) = Φ ( {v}_{1} , {v}_{2} ) / \sqrt{Φ ( {v}_{1} , {v}_{1} ) \cdot Φ ( {v}_{2} , {v}_{2} )}$$
$$ξ ( {v}_{1} , {v}_{2} ) = Ψ ( {v}_{1} , {v}_{2} ) / \sqrt{Φ ( {v}_{1} , {v}_{1} ) \cdot Φ ( {v}_{2} , {v}_{2} )}$$
where:
$${ρ ( {v}_{1} , {v}_{2} )}^{2} + {ξ ( {v}_{1} , {v}_{2} )}^{2} = 1 .$$
